(19) United States
(12) Patent Application Publication
(10) Pub. No.: US 2021/0022841 A1
(43) Pub. Date: Jan. 28, 2021

(54) TRANSVAGINAL TREATMENT OF STRESS URINARY INCONTINENCE

(71) Applicant: Covidien LP, Mansfield, MA (US)

(72) Inventors: Nikolai D. Begg, Wellesley, MA (US); Chad A. Pickering, Woburn, MA (US); Rebecca D. White, Kennett Square, PA (US); Lisa M. Quealy, Dracut, MA (US); Jordan A. Whisler, Brookline, MA (US)

(21) Appl. No.: 16/936,744

(22) Filed: Jul. 23, 2020

Related U.S. Application Data
(60) Provisional application No. 62/877,586, filed on Jul. 23, 2019.

(51) Int. Cl. A61F 2/00; A61B 17/068; A61B 17/42

(52) U.S. Cl. CPC A61F 2/0031; A61B 2017/0647; A61B 17/42; A61B 17/068

(57) ABSTRACT

A method of treating stress urinary incontinence includes moving a vaginal fornix of a female patient adjacent to a pectineal ligament of the female patient and securing the vaginal fornix to the pectineal ligament. The method may include inserting a fastener applier into a female patient to secure the vaginal fornix to the pectineal ligament. The method may include positioning a fastener applier adjacent to the vaginal fornix before moving the vaginal fornix.

### CROSS-REFERENCE TO RELATED APPLICATIONS

This application claims the benefit of U.S. Provisional Patent Application No. 62/877,586, filed Jul. 23, 2019, the entire contents of which are incorporated by reference herein.

### TECHNICAL FIELD

This disclosure relates to surgical methods, and more particularly, to transvaginal treatment of stress urinary incontinence.

### BACKGROUND

Stress urinary incontinence (SUI) occurs when urine leaks out with sudden pressure on the bladder and urethra, causing the sphincter muscles to open briefly. With mild SUI, pressure may be from sudden forceful activities, like exercise, sneezing, laughing or coughing. SUI is a very common bladder problem for women. In particular, about 1 in 3 women suffer from SUI at some point in their lives. It happens less often in men.

### SUMMARY

The disclosure generally relates to treatment of stress urinary incontinence.

In accordance with an aspect, this disclosure relates to a method of treating stress urinary incontinence. The method includes moving a vaginal fornix of a female patient adjacent to a pectineal ligament of the female patient and securing the vaginal fornix to the pectineal ligament.

In aspects, the method may further include inserting a fastener applier into a female patient to secure the vaginal fornix to the pectineal ligament. Inserting the fastener applier into the female patient may be performed transvaginally. Inserting the fastener applier may include inserting a tack applier.

In various aspects, securing the vaginal fornix to the pectineal ligament may include fastening the vaginal fornix to the pectineal ligament with a fastener.

In aspects, securing the vaginal fornix to the pectineal ligament may include fastening the vaginal fornix to the pectineal ligament with a tack. Fastening the vaginal fornix to the pectineal ligament with a tack may include firing the tack from a tack applier.

In various aspects, the method may involve positioning a fastener applier adjacent to the vaginal fornix before moving the vaginal fornix. Moving the vaginal fornix may include elevating the vaginal fornix toward the pectineal ligament of the female patient with the fastener applier to reduce a width of a urethral channel of a urethra of the female patient.

In aspects, moving the vaginal fornix of the female patient adjacent to the pectineal ligament may include moving an anterior vaginal fornix of the female patient adjacent to the pectineal ligament. Securing the vaginal fornix to the pectineal ligament may include securing the anterior vaginal fornix to the pectineal ligament.

The details of one or more aspects of this disclosure are set forth in the accompanying drawings and the description below. Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a side view illustrating anatomy of a central portion of a female patient having stress urinary incontinence (only relevant anatomy shown for clarity);

FIG. 2 is a top view of the anatomy illustrated in FIG. 1;

FIG. 3 is a side view of the anatomy illustrated in FIG. 1 with a fastener applier shown inserted into an anterior fornix of the female patient;

FIGS. 4-6 are progressive views illustrating the fastener applier securing the anterior fornix of the female patient to a pectineal ligament of the female patient with a fastener of the fastener applier;

FIG. 7 is a side view of a bladder and urethra of the female patient before the anterior fornix of the female patient is secured to the pectineal ligament of the female patient, the urethra disposed in an open position; and

FIG. 8 is a side view of the bladder and urethra of the female patient after the anterior fornix of the female patient is secured to the pectineal ligament of the female patient, the urethra disposed in a closed position.

### DETAILED DESCRIPTION

Aspects of this disclosure are described in detail with reference to the drawings, in which like reference numerals designate identical or corresponding elements in each of the several views. As commonly known, the term “clinician” refers to a doctor (e.g., a surgeon), a nurse, or any other care provider and may include support personnel. Additionally, the term “proximal” refers to the portion of structure that is closer to the clinician and the term “distal” refers to the portion of structure that is farther from the clinician. In the following description, well-known functions or constructions are not described in detail to avoid obscuring this disclosure in unnecessary detail.

In general, this disclosure describes a method for treating stress urinary incontinence including inserting a fastener applier, such as a curved tacking device, into an anterior vaginal fornix and elevating the anterior vaginal fornix for securing the anterior vaginal fornix to a pectineal ligament with a fastener, such as a tack, of the fastener applier. The fastener fixes the vaginal fornix to the pectineal ligament to support and suspend a urethra of the patient in a manner sufficient to reduce stress urinary incontinence of the patient.

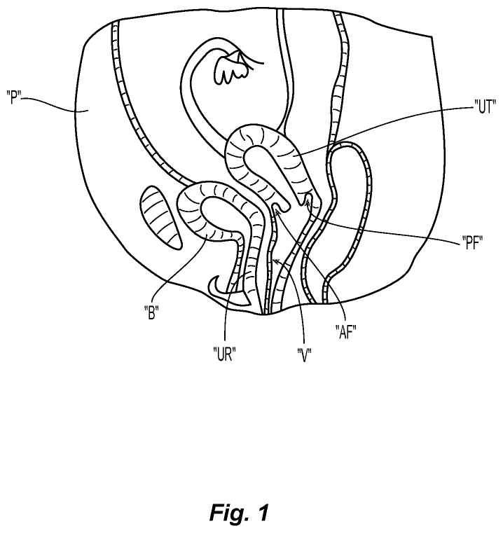

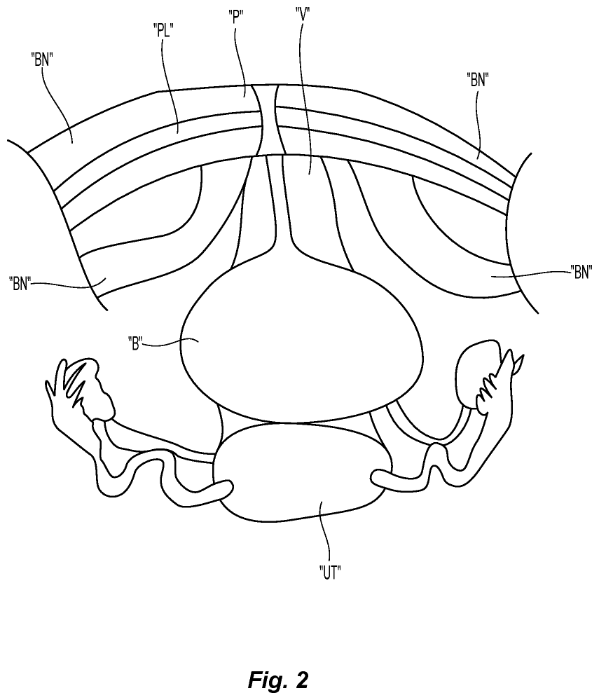

FIGS. 1 and 2 illustrate anatomy of a female patient “P.” For instance, FIG. 1, which is a side view of a central portion of the female patient “P,” delineates the patient's bladder “B,” urethra “UR,” vagina “V,” as well as anterior and posterior vaginal fornix “AF,” “PF,” respectively. FIG. 2, which is a top view of the central portion of the female patient “P,” delineates the patient's bladder “B”, uterus “UT,” bone “BN”, and pectineal ligament “PL.”

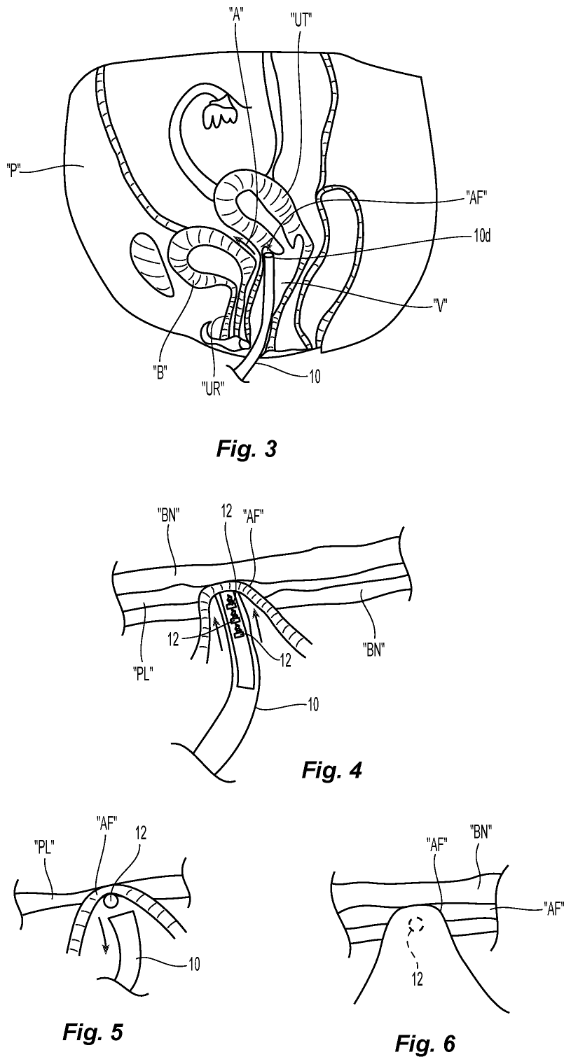

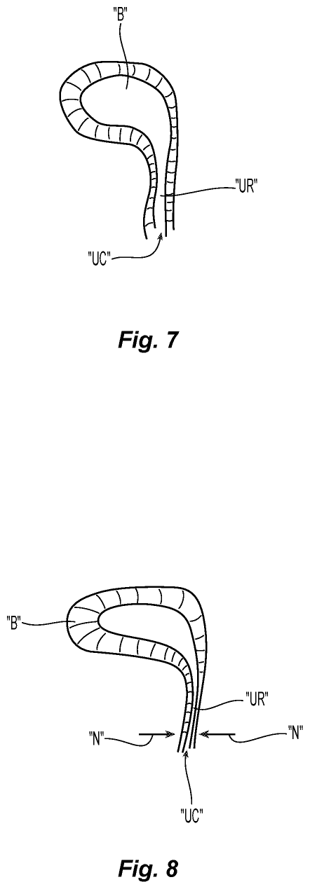

Turning now to FIGS. 3-8, to treat stress urinary incontinence, a clinician can insert a fastener applier 10 into the patient's vagina “V,” for example, transvaginally as seen in FIG. 3. Fastener applier 10 can be a tack applier supporting one or more fasteners (e.g., tacks) 12. The fastener applier 10 may be a curved fastener applier, a flexible fastener applier, and/or an articulating fastener applier. For a more detailed description of such fastener appliers, reference can be made, for example, to U.S. Pat. No. 10,085,746 to Fischvogt et al. or to U.S. Pat. No. 9,358,010 to Wenchell et al., the entire contents of each of which are incorporated by reference herein.

Fastener applier 10 is advanced through vagina “V” until a distal end portion 10d of fastener applier 10 is engaged with the anterior vaginal fornix “AF” of the patient “P.” The clinician then elevates the anterior vaginal fornix “AF” with fastener applier 10, by advancing fastener applier 10 further into patient “P.” The clinician then guides the vaginal fornix “AF” toward the pectineal ligament “PL,” as indicated by arrow “A.” Once the vaginal fornix “AF” is disposed adjacent to the pectineal ligament “PL,” the clinician can actuate fastener applier 10 to fire one or more fasteners 12 from the fastener applier 10 to secure the vaginal fornix “AF” to the pectineal ligament “PL” by the one or more fasteners 12. Securing the vaginal fornix “AF” to the pectineal ligament “PL” tightens the urethra “UR” and narrows a urethra channel “UC” defined by the urethra “UR,” as indicated by arrows “N” (FIG. 8) to limit urine leakage associated with stress urinary incontinence. The fastener applier 10 can then be withdrawn.

The various aspects disclosed herein may also be provided in connection with robotic surgical systems and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients. For a detailed description of exemplary medical work stations and/or components thereof, reference may be made to U.S. Pat. No. 8,828,023, and PCT Application Publication No. WO2016/025132, the entire contents of each of which are incorporated by reference herein.

Persons skilled in the art will understand that the structures and methods specifically described herein and shown in the accompanying figures are non-limiting exemplary aspects, and that the description, disclosure, and figures should be construed merely as exemplary of particular aspects. It is to be understood, therefore, that this disclosure is not limited to the precise aspects described, and that various other changes and modifications may be effected by one skilled in the art without departing from the scope or spirit of this disclosure. Additionally, the elements and features shown or described in connection with certain aspects may be combined with the elements and features of certain other aspects without departing from the scope of this disclosure, and that such modifications and variations are also included within the scope of this disclosure. Accordingly, the subject matter of this disclosure is not limited by what has been particularly shown and described.

What is claimed is:

1. A method of treating stress urinary incontinence, the method comprising:
moving a vaginal fornix of a female patient adjacent to a pectineal ligament of the female patient; and
securing the vaginal fornix to the pectineal ligament.

2. The method of claim 1, further comprising:
inserting a fastener applier into a female patient to secure the vaginal fornix to the pectineal ligament.

3. The method of claim 2, wherein inserting the fastener applier into the female patient is performed transvaginally.

4. The method of claim 3, wherein inserting the fastener applier includes inserting a tack applier.

5. The method of claim 2, wherein securing the vaginal fornix to the pectineal ligament includes fastening the vaginal fornix to the pectineal ligament with a fastener.

6. The method of claim 2, wherein securing the vaginal fornix to the pectineal ligament includes fastening the vaginal fornix to the pectineal ligament with a tack.

7. The method of claim 6, wherein fastening the vaginal fornix to the pectineal ligament with a tack includes firing the tack from a tack applier.

8. The method of claim 1, further comprising positioning a fastener applier adjacent to the vaginal fornix before moving the vaginal fornix.

9. The method of claim 8, wherein moving the vaginal fornix includes elevating the vaginal fornix toward the pectineal ligament of the female patient with the fastener applier to reduce a width of a urethral channel of a urethra of the female patient.

10. The method of claim 1, wherein moving the vaginal fornix of the female patient adjacent to the pectineal ligament includes moving an anterior vaginal fornix of the female patient adjacent to the pectineal ligament, and wherein securing the vaginal fornix to the pectineal ligament includes securing the anterior vaginal fornix to the pectineal ligament.
